(19) United States
(12) Patent Application Publication
(10) Pub. No.: US 2012/0016182 A1
(43) Pub. Date: Jan. 19, 2012

(54) Fold-Resistant Pelvic Implant System and Method

(75) Inventors: Guangjian Wang, Falcon Heights, MN (US); Jose' W. Jimenez, Apple Valley, MN (US); Edouard A. Koullick, Golden Valley, MN (US)

(73) Assignee: AMS Research Corporation, Minnetonka, MN (US)

(21) Appl. No.: 13/182,858

(22) Filed: Jul. 14, 2011

Related U.S. Application Data
(60) Provisional application No. 61/364,225, filed on Jul. 14, 2010.

(51) Int. Cl. A61F 2/04

(52) U.S. Cl. 600/30; 600/37

(57) ABSTRACT

A pelvic non-folding hammock implant is provided. Such an implant can be constructed of a polymer material to provide an implant portion, e.g., support portion, having one or more extending and independent teeth-like features defining a fold control portion in a generally comb-like construct. The individual teeth resist folding by providing independently reacting extensions along the fold control portion.

### PRIORITY

This Application claims priority to and the benefit of U.S. Provisional Application No. 61/364,225, filed Jul. 14, 2010, which is incorporated herein by reference in its entirety.

### FIELD OF THE INVENTION

The present invention relates generally to surgical methods and apparatus and, more specifically, to surgically implantable patterned support devices and methods for forming and using the same.

### BACKGROUND OF THE INVENTION

Pelvic health for men and women is a medical area of increasing importance, at least in part due to an aging population. Examples of common pelvic ailments include incontinence (fecal and urinary), pelvic tissue prolapse (e.g., female vaginal prolapse), and conditions of the pelvic floor.

Urinary incontinence can further be classified as including different types, such as stress urinary incontinence (SUI), urge urinary incontinence, mixed urinary incontinence, among others. Other pelvic floor disorders include cystocele, rectocele, enterocele, and prolapse such as anal, uterine and vaginal vault prolapse. A cystocele is a hernia of the bladder, usually into the vagina and introitus. Pelvic disorders such as these can result from weakness or damage to normal pelvic support systems.

Urinary incontinence can be characterized by the loss or diminution in the ability to maintain the urethral sphincter closed as the bladder fills with urine. Male or female stress urinary incontinence (SUI) generally occurs when the patient is physically stressed. Physical stresses that can cause urinary incontinence include jumping, coughing, sneezing and laughing to name a few.

In its severest forms, vaginal vault prolapse can result in the distension of the vaginal apex outside of the vagina. An enterocele is a vaginal hernia in which the peritoneal sac containing a portion of the small bowel extends into the rectovaginal space. Vaginal vault prolapse and enterocele represent challenging forms of pelvic disorders for surgeons. These procedures often involve lengthy surgical procedure times.

Many strategies have been implemented over the years to provide mesh implants adapted to enhance therapeutic support of the respective pelvic tissues. For instance, sling and other implant devices are known to provide support of the urethra or bladder neck in treating urinary incontinence in patients. Further, various mesh implants have been adapted to provide pelvic floor support to treat certain prolapse disorders.

Many of the implants promoted for treating incontinence, prolapse and other pelvic disorders were born from and inherited the material and geometric restraints of existing stent and hernia implants. While objectively effective in their respective applications, such devices can fold or otherwise undesirably deform during and after the implantation procedure.

Although these traditional mesh implants have had a tremendous benefit for those suffering from incontinence and prolapse, there is still room for improvement. As a result, there is a desire to obtain a uniquely applicable, minimally invasive and highly effective implantable support that can be used to treat incontinence (urinary or fecal), organ prolapse and other pelvic disorders and conditions.

### SUMMARY OF THE INVENTION

The present invention describes implants and methods for treating pelvic conditions such as incontinence (various forms such as fecal incontinence, stress urinary incontinence, urge incontinence, mixed incontinence, etc.), vaginal prolapse (including various forms such as enterocele, cystocele, rectocele, apical or vault prolapse, uterine descent, etc.), and other conditions caused by muscle or ligament weakness. Other uses include providing a support or platform for plastic surgery, hernia repair, and ortho repairs and support, to name a few. Embodiments of the implants can include a tissue support portion and one or more extending arms or anchoring portions.

In various embodiments, the implants can be formed or otherwise provided from a woven porous mesh, or from molding, die casting, laser etching, laser cutting, extruding, and the like (porous or non-porous). Such an implant can be constructed of a polymer material to provide an implant portion, e.g., support portion, having one or more extending and independent, distinct or separate finger members or teeth-like features defining a fold control portion in a generally comb-like construct. Portions of the implant, other than the support portion, can also include the extending teeth features.

The inclusion of one or more extending teeth features provides a support system that is non-foldable, or substantially non-foldable. The individual teeth of the features are more resistant to folding than a standard mesh or other material implant as a whole. The individual teeth are generally discontinuous or independent from one another such that folding or deformation of one tooth feature will not affect another tooth in the comb-like construct.

Each individual tooth of the fold control portion can be provided in a nearly endless array of shape and size options, including rounded, squared, tapered, jagged, straight, undulating, curved, and the like. Further, the teeth of the fold control portion can be thicker, thinner, stiffer, or more flexible than the dimensions of the remaining implant portions. The overall footprint of the fold control portion can take on or define a myriad of shapes as well, including elliptical, oval, circular, rectangular, square, and the like. Various shapes and designs can be implemented depending on the anatomical location and shape of the target tissue area.

In addition, each individual extending tooth, or the overall teeth construct, can include uniquely shaped or cut members configured to optimize stability, promote tissue in-growth, promote load bearing along select portions of the implant, and to generally provide focused control of stiffness, elongation and compression.

In addition to molding and laser cutting the struts and other features of the implant, punching, 3-D printing and other methods and techniques can be employed in making the implant. Further, the fold control portion, or other portions of the implant, can be coated to provide additional control over expansion, folding, compression, and to protect from or promote tissue in-growth.

### BRIEF DESCRIPTION OF THE DRAWINGS

FIG. 1 is a top view of a pelvic hammock or implant having a fold control portion and anchors in accordance with embodiments of the present invention.

FIG. 2 is a top view of a fold control portion of a pelvic hammock or implant in accordance with embodiments of the present invention.

FIGS. 3-4 are top views of a fold control portion of a pelvic hammock or implant illustrating a general profile shape in accordance with embodiments of the present invention.

FIG. 5 is a top view of a fold control portion of a pelvic hammock or implant in accordance with embodiments of the present invention.

FIG. 6 is a perspective view of a delivery or introduction tool, having handle actuation, for use with embodiments of the present invention.

FIG. 7 is a side view of a delivery or introduction tool for use with embodiments of the present invention.

### DETAILED DESCRIPTION OF PREFERRED EMBODIMENTS

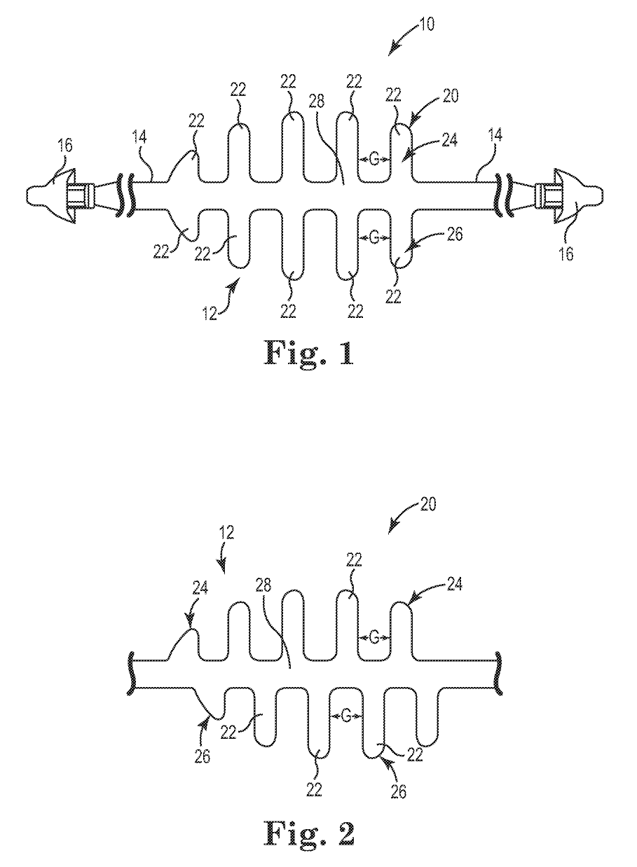

Referring generally to FIGS. 1-7, various embodiments of a generally non-folding pelvic hammock or implant 10 system and method are shown. In general, the implants 10 can include a tissue support portion 12 and tissue anchoring portions 16. An extension portion 14 can be included to span between or link the support portion 12 and the respective anchoring portions 16. Various portions of the implant 10 can be constructed of polymer materials, e.g., woven, shaped, molded or otherwise formed into or from a generally planar film or sheet material. Examples of acceptable polymer materials available in constructing or forming the implant systems 10 and its components can include polypropylene, polyethylene, fluoropolymers or like biocompatible materials.

The various implants 10, structures, features and methods detailed herein are envisioned for use with many known implant and repair devices (e.g., for male and female urinary and fecal incontinence solutions), features and methods, including those disclosed in U.S. Pat. Nos. 7,500,945, 7,407,480, 7,351,197, 7,347,812, 7,303,525, 7,070,556, 7,025,063, 6,911,003, 6,802,807, 6,702,827, 6,691,711, 6,652,450, 6,648,921, and 6,612,977, International Patent Publication Nos. WO 2011/072148, WO 2008/057261 and WO 2007/097994, and U.S. Patent Publication Nos. 2011/0124956, 2010/0261955, 2004/0039453, 2002/0151762 and 2002/0147382. Accordingly, the above-identified disclosures are fully incorporated herein by reference in their entirety.

Referring generally to FIGS. 1-5, various embodiments of the hammock or implant 10 are shown. Portions of the implant 10, such as the support portion 12, can be formed of a mesh material (woven or non-woven), or formed or patterned by way of a polymer molding process to create a unitary generally homogeneous non-woven, or non-knitted, device or construct. Other embodiments can be formed from an already unitary homogeneous sheet or film via laser cutting, die cutting, stamping and like procedures. Further, various embodiments of the implant 10 can be constructed of opaque, or translucent, polymer materials. The support portion 12 is generally adapted to support tissue, such as that required to treat urinary or fecal incontinence, including the bladder neck, urethra or rectum.

A fold control or resistant portion 20 is included with the implant 10, such as the support portion 12. The fold control portion 20 can define a generally comb-like construct including one or more finger or teeth features 22. The teeth features 22 can include a plurality of teeth features 22 provided in various configurations, shapes, sizes and the like. In various embodiments, the plurality of teeth 22 can be separated and distinct from one another and provided in opposing rows 24, 26 flanking, or separated by, an intermediate portion 28. The intermediate portion 28 generally extends along a longitudinal axis of the implant 10. The plurality of teeth 22 extending generally transverse to the intermediate portion 28 and the implant longitudinal axis. However, the teeth 22 can be configured to extend from the intermediate portion 28 along any angle or position without deviating from the invention.

As shown in FIGS. 1 and 4, the opposing extending teeth features 22 of rows 24, 26 can be generally aligned such that gaps G are defined between the features 22 and are also generally aligned in the opposing rows 24, 26. Other embodiments, such as those presented in FIGS. 2 and 5, can include extending teeth features 22 generally offset from opposing features 22 in rows 24, 26. These offset configurations of the fold control portion 20 provide a construct including teeth 22 generally aligned with corresponding gaps G in the opposing row (FIG. 2).

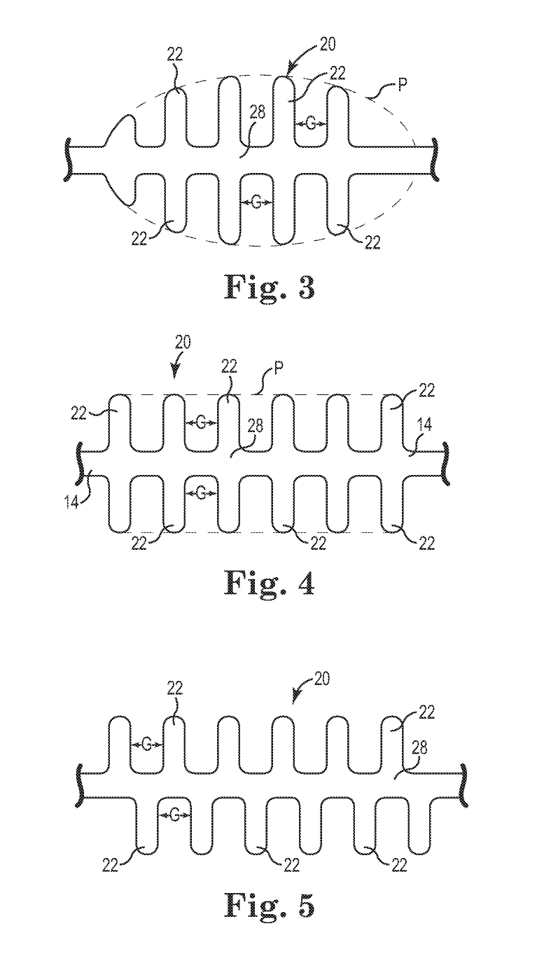

FIG. 3 depicts the embodiment of FIG. 1 with a profile or perimeter phantom line P illustrating the generally elliptical or oval shape of the fold control portion 20 as a whole. This particular shape of the portion 20 can be accomplished by including teeth 22 at differing lengths, shapes and like dimensional configurations. FIG. 4 depicts other embodiments with a perimeter phantom line P illustrating a generally rectangular or box shaped fold control portion 20, as a whole. The fold control portion 20 can take on a myriad of alternative sizes, shapes and footprint configurations, including square, undulating, triangular, circular, and the like.

Further, each individual extending tooth 22, or its distal tip regions, can be provided in a nearly endless array of shape and size options, including rounded, squared, tapered, jagged, undulating, straight, angled, curved, and the like. Moreover, the teeth 22 of the fold control portion can be thicker, thinner, stiffer, or more flexible than the properties or characteristics of the remaining implant 10 portions, including the intermediate portion 28.

Each individual extending tooth or finger 22, or the overall portion 20 construct, can include uniquely shaped or cut members configured to optimize stability, promote tissue in-growth, promote load bearing along select portions of the implant, and to generally provide focused control of folding, stiffness, elongation and compression.

Embodiments of the implant 10 can include one or more transition portions or zones, thereby providing a material transition between the fold control portion 20 and the extending portion 14 and/or the anchor portion 16. The transition zones can take on various sizes, shapes and designs (e.g., mesh, barrels, rods, etc.) to provide increased strength and stress absorption/distribution for portions (e.g., portion 20) of the implant 10 being pulled, pushed and twisted during deployment and positioning of the implant 10.

The structure and design of anchoring features of portions 16 of the implant 10 can vary greatly depending on the particular implantation and support needs of the particular device. In certain embodiments, the anchor portions 16 can include first and second opposing end anchors extending out from the extension portions 14 of the implant 10, and including one or more tissue engaging tines or barbs.

By providing the size, spacing, shape and general configuration of the teeth 22 along the rows 24, 26 of the portion 20, the extending teeth features 22 provide a support system that is non-foldable, or substantially non-foldable. The individual teeth of the features are more resistant to folding than a standard mesh or other material implant as a whole. The individual teeth 22 are generally discontinuous or independent from one another such that folding or deformation of one tooth 22 will not affect another adjacent or opposing tooth 22 in the comb-like construct 20. As such, pressure or other forces on a select tooth 22 that may cause of level of collapse, deformation or even folding will remain isolated such that the remainder of the teeth 22 are unaffected and the portion 20 as a whole remains generally free of folding, deformation and the like. The intermediate portion 28 and the teeth features 22 can be constructed of the same or different materials or flexibility configurations to facilitate this isolation and individual reaction feature.

More than one material can be used to construct the implant 10 or its portions (e.g., fold control portion 20) to further control the desired properties and characteristics described herein, e.g., combining different polymers such as polypropylene, PEEK, PET, PTFE, PGA, PLA, etc. Polymers could also be combined with metallic elements to alter strength, stiffness and flexibility properties of the implant 10.

The implants 10 described herein can be implanted into a patient by use of various different types of surgical tools, including insertion tools, which generally are tools useful to engage and place a tissue anchor or a connector that is secured to an extension portion of an implant. Various types of insertion tools are known, including those in the previously-incorporated references, and these types of tools and modifications thereof can be used according to the present description to install the implant 10.

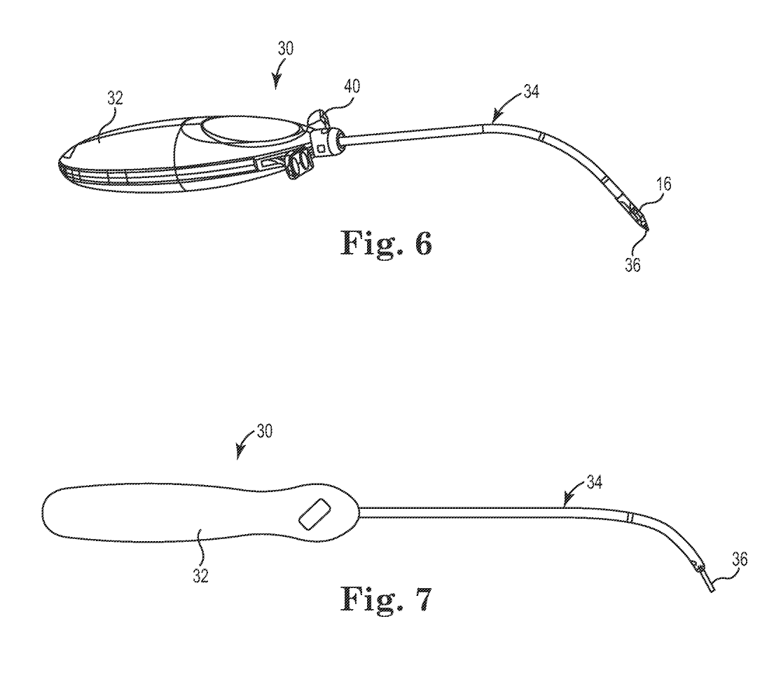

Examples of various insertion techniques and tools are included in FIGS. 6-7. Each tool 30 can include a handle 32, needle 34 and engaging distal tip 36. The handle 32 can include an actuation mechanism 40 in operative communication with the distal tip 36 and adapted to selectively control engagement and/or disengagement of the distal tip 36 with portions of the implant 10 (e.g., anchors 16).

The implants 10, their various components, portions, structures, features, materials and methods may have a number of suitable configurations and applications, as shown and described in the previously-incorporated references. Various methods and tools for introducing, deploying, anchoring and manipulating implants to treat incontinence and prolapse as disclosed in the previously-incorporated references are envisioned for use with the present invention as well.

All patents, patent applications, and publications cited herein are hereby incorporated by reference in their entirety as if individually incorporated, and include those references incorporated within the identified patents, patent applications and publications.

Obviously, numerous modifications and variations of the present invention are possible in light of the teachings herein. It is therefore to be understood that within the scope of the appended claims, the invention may be practiced other than as specifically described herein.

1. A hammock implant device for treating a pelvic disorder, comprising:
a support portion including a plurality of extending teeth separated by a spacing gap; and
first and second opposing tissue anchors in operable communication with the support portion.

2. The implant device of claim 1, further including opposing extension portions extending between the support portion and respective first and second tissue anchors.

3. The implant device of claim 1, wherein the plurality of extending teeth includes two separate rows of extending teeth flanking an intermediate portion.

4. The implant device of claim 3, wherein the extending teeth are substantially aligned in the two separate rows.

5. The implant device of claim 3, wherein the extending teeth in the two separate rows are in offset alignment.

6. The implant device of claim 1, wherein at least the support portion is constructed of a non-porous material.

7. The implant device of claim 1, wherein at least the support portion is constructed of a porous mesh material.

8. The implant device of claim 1, wherein the footprint profile of the support portion is generally rectangular.

9. The implant device of claim 1, wherein the footprint profile of the support portion is generally elliptical.

10. The implant device of claim 1, wherein end portions of the plurality of teeth are generally rounded.

11. The implant device of claim 1, wherein end portions of the plurality of teeth are generally angled.

12. The implant device of claim 1, wherein the support portion is adapted for positioning to treat fecal incontinence.

13. The implant device of claim 1, wherein the support portion is adapted for positioning to treat urinary incontinence.

11. An implant device for treating a pelvic disorder, comprising:
a tissue support portion including;
a fold control portion having a first plurality of extending finger members and a second plurality of extending finger members; and
an intermediate portion, wherein the first plurality of extending finger members extend in a first direction from the intermediate portion and the second plurality of extending finger members extend from the intermediate portion in a direction generally opposite the first direction.

12. The implant device of claim 11, wherein the first and second extending finger members are substantially in opposing alignment.

13. The implant device of claim 11, wherein the first and second extending finger members are generally offset.

14. The implant device of claim 11, wherein the support portion is constructed of a non-porous material.

15. The implant device of claim 11, wherein the support portion is constructed of a porous mesh material.

16. The implant device of claim 11, wherein the fold control portion defines a generally rectangular perimeter profile.

17. The implant device of claim 11, wherein the fold control portion defines a generally elliptical perimeter profile.

18. The implant device of claim 11, wherein end portions of at least the first plurality of extending finger members are generally rounded.

19. The implant device of claim 11, wherein end portions of at least the first plurality of extending finger members are generally angled.

20. The implant device of claim 11, wherein at least two adjacent extending finger members in the first and second plurality of extending finger members include a gap spacing therebetween.
